(19) United States
(12) Patent Application Publication
(10) Pub. No.: US 2022/0264876 A1
(43) Pub. Date: Aug. 25, 2022

(54) COMPOSITIONS AND METHODS FOR ENHANCING PLANT GROWTH

(71) Applicant: BOYCE THOMPSON INSTITUTE FOR PLANT RESEARCH, INC., Ithaca, NY (US)

(72) Inventors: Daniel KLESSIG, Dryden, NY (US); Murli MANOHAR, Ithaca, NY (US); Frank SCHROEDER, Ithaca, NY (US)

(21) Appl. No.: 17/621,927

(22) PCT Filed: Jul. 8, 2020
(86) PCT No.: PCT/US20/41132

§ 371 Date: Dec. 22, 2021

Related U.S. Application Data
(60) Provisional application No. 62/871,486, filed on Jul. 8, 2019.

(51) Int. Cl. A01N 43/16; A01N 3/04

(52) U.S. Cl. CPC A01N 43/16; A01N 3/04

(57) ABSTRACT

Compositions and methods for promoting, enhancing, and/or increasing plant growth are disclosed.

This application claims priority under 35 U.S.C. § 119(e) to U.S. Provisional Patent Application No. 62/871,486, filed Jul. 8, 2019. The foregoing application is incorporated by reference herein.

This invention was made with government support under Grant No. 12217687 awarded by the National Institute of Food and Agriculture. The government has certain rights in the invention.

### FIELD OF THE INVENTION

This invention relates to the fields of agriculture. More specifically, the invention provides methods and compositions for the enhancement of plant growth.

### BACKGROUND OF THE INVENTION

The rapidly increasing human population, expected to reach 9.5-10 billion by 2050, coupled with changing climate is accentuating the ever-increasing need to enhance food production. Sustainable production of sufficient food and fiber from the earth's limited resources, including fresh water and land, is paramount for food security and long-term international order. The challenges have and continue to be addressed using improved, higher yielding plants often with increased resistance to biotic (e.g. pest) and abiotic (e.g. droughts) stresses, improved cultivation and management practices (e.g. crop rotation), and plant growth enhancers (PGEs). PGEs can contain organic and inorganic components and be of natural on synthetic origin. The most common are fertilizers contain nitrogen, phosphorous and potash. The importance of living microorganisms as highly beneficial PGE is becoming increasingly evident, particular in light of limited resources, sustainability, and environmental health. Two of the best studied are symbiotic mycorrhizal fungi, which help plants access nutrients, particularly phosphate, from the soil (Bonfante et al. (2009) Annu. Rev. Microbiol., 63:363-383), and growth promoting rhizobacteria, which help plants tolerate abiotic stresses (Etesami, et al. (2010) Ann. Microbiol., 60:579-598). However, despite the current array of PGEs, the need to further increase food and fiber production in a sustainable environmentally friendly manner demands new and improved approaches to enhance yield. Against this background, the present invention provides effective methods and compositions to enhanced plant growth.

### SUMMARY OF THE INVENTION

In accordance with the instant invention, method for promoting, enhancing, and/or increasing plant growth are provided. In a particular embodiment, the method comprises contacting the target plant and/or its environment with a compound with a chemical structure identical to a compound produced by a pathogen. In a particular embodiment, the pathogen is a nematode. The compound produced by the pathogen may comprise an ascaroside (e.g., ascr #18). In a particular embodiment, the method comprises contacting the target plant and/or its environment with an ascaroside (e.g., ascr #18) and/or a side-chain shortened analog or metabolite of the ascaroside (e.g., a side-chain shortened analog of ascr #18 such as ascr #9, ascr #10, and/or ascr #1). The compounds may be applied to any part of the plant (e.g., seed, root, and/or foliage) and/or the nearby soil. The methods of the instant invention may be effective to result in one or more (or all) of the following: i) increased speed and/or consistency of germination, ii) increased rate of growth of the roots, shoots, and/or plant (e.g., height), iii) reduced time to flowering and/or seed set, and iv) increased yield such as amount/weight of grain, weight of tubers, and/or total biomass. The compounds of the instant invention may be delivered to the plant in a composition further comprising a carrier such as an agronomically acceptable carrier. In a particular embodiment, the composition is a liquid composition and is sprayed onto the plant or plant part. In a particular embodiment, the composition is coated onto the plant or plant part (e.g., seed). In a particular embodiment, the compounds are delivered to the plant in a sterile or nearly sterile environment (e.g., in the general absence of pathogens (e.g., nematode, virus, bacteria, fungus, insect, and/or oomycete, particularly in the absence of nematodes and bacteria).

### BRIEF DESCRIPTION OF THE DRAWINGS

FIGS. 1A-1D show the effect of ascr #18 on rice seed germination/emergence and growth. Rice seeds were pretreated 24 hours without (mock) or with different concentrations (10, 100 and 1000 nM) of ascr #18 before planting in soil. Seed germination/emergence from soil was measured after 7, 8, 9, 10 and 11 days after planting (FIG. 1A). The length of the plants was measured 15 days after planting (FIG. 1B) and 18 days after planting (FIG. 1C). Pictures were taken 15 days after planting (FIG. 1D). Data are averages±s.d. (n≥12). ***P≤0.0005; ****P≤0.00005; two-tailed t-test.

FIGS. 2A-2C show the effect of ascr #18 on rice seed germination/emergence and growth. Rice seeds were pretreated 24 hours without (mock) or with different concentrations (10, 100 and 1000 nM) of ascr #18 before planting in soil. Seed germination/emergence from soil was measured after 6, 7, 8, 9 and 10 days after planting (FIG. 2A). The length of the plants was measured 10, 12, 14, 15 and 18 days after planting (FIG. 2B). The fresh biomass of the plants was measured 18 days after planting (FIG. 2C). Data are averages±s.d. (n≥14). *P≤0.05; **P≤0.005; ***P≤0.0005; ****P≤0.00005; two-tailed t-test.

FIG. 3 shows the effect of ascr #18 on tomato plant growth. Tomato seeds were pretreated 24 hours without (mock) or with different concentrations (1, 10, 100, 1000 and 10000 nM) of ascr #18 before planting on soil. The length of the plants was measured 12 days after planting. **P≤0.002; ****P≤0.00005; two-tailed t-test.

FIGS. 4A-4F show the effect of ascr #18 on tomato seed germination/emergence and growth. Tomato seeds were pretreated 24 hours without (mock) or with different concentrations (10, 100, and 1000 nM) of ascr #18 before planting in soil. Seed germination/emergence from soil was measured after 4, 5, 6 and 7 days after planting (FIG. 4A). The length of plants was measured 12 and 14 days after planting (FIG. 4B). Pictures were taken 14 days after planting (FIG. 4C). **P≤0.002; ****P≤0.00005; two-tailed t-test. FIGS. 4D and 4E provide graphs of the fresh weight of above-ground (excluding root) part of the plants measured at day 21 after planting in soil for tomato seeds pretreated without (mock) or with different concentrations (0.1 and 10 μM) of ascr #18 for 24 hours (FIG. 4D) or 72 hours (FIG. 4E) before planting in soil. The length of the roots of the plants was also measured 21 days after planting (FIG. 4F).

FIGS. 5A-5D show that ascr #18 stimulates germination and growth in Arabidopsis. Arabidopsis seeds were pretreated 24 hours without (mock) or with 1 μM ascr #18 before planting in soil. FIG. 5A provides a graph of the number of plants at different stages of germination counted 3 days after planting. Stages of germination; 0=no germination; 1=about to germinate; 2=germinated and two-leaf stage; 3=germinated and have expanded leaves (n=60). FIG. 5B provides graphs of the surface area of germinated plants measured by taking pictures and counting pixels using Fiji software and then normalizing by the total area for each plant (n=15 pots with each pot containing 4 plants). FIG. 5C provides a graph of the percent of plants flowering at day 25 after planting in soil. FIG. 5D provides a graph of the fresh weight of above-ground (excluding root) part of the plants measured at day 25 after planting in soil. *P=0.03; **P≤0.01; ***P≤0.001; ****P≤0.0001; two-tailed t-test.

FIGS. 6A-6B show the effects of ascr #18 on potato plants. Four-week old potato plants were pretreated without (mock) or with ascr #18 (10 nM). Potato tubers were harvested from mock and pre-treated plants. FIG. 6A provides images of tubers from one representative plant that was mock treated and one representative plant that was treated with ascr #18. FIG. 6B provides a graph of the biomass of the above-ground (excluding root) part of the potato plants.

FIGS. 7A-7E show the effect of ascr #18 on potato plant growth and yield. Four-week old potato plants were sprayed once with buffer only (mock) or 10 nM ascr #18 before harvesting two weeks later. FIG. 7A shows images of representative plants two weeks after treatment with either mock solution or ascr #18. FIG. 7B shows the above-ground biomass of treated plants at harvest. Potato root system (7C) and tubers (7D) harvest from treated and mock plants are presented. Each pile of tubers was from one plant. FIG. 7E presents the tuber biomass of the mock and treated plants at harvest.

FIGS. 8A-8B show the effect of ascr #18 on corn seed germination/emergence and growth. Corn seeds were pretreated 24 hours without (mock) or with different concentrations (10, 100 and 1000 nM) of ascr #18 before planting in soil. Seed germination/emergence from soil was measured after 4, 5, 6, 7, 8, 9, 10 and 11 days after planting (FIG. 8A). The height of the second node of the plants was measured 15 days after planting (FIG. 8B). Data are averages±s.d. (n≥12). *P≤0.05; two-tailed t-test.

FIGS. 9A-9B show the effect of ascr #18 on wheat seed germination/emergence and growth. Wheat seeds were pretreated 24 hours without (mock) or with different concentrations (10, 100 and 1000 nM) of ascr #18 before planting in soil. Seed germination/emergence from soil was measured after 2, 3, 4, 5, 6, 7, 8, 9 and 10 days after planting (FIG. 9A). The height of the second node of the plants was measured 15 days after planting (FIG. 9B). Data are averages±s.d. (n≥12). *P≤0.05; two-tailed t-test.

### DETAILED DESCRIPTION OF THE INVENTION

Nematode ascarosides (NAs) form a highly-conserved family of nematode-derived small signaling molecules. Some NAs, including ascr #18, induce hallmark plant defenses including activation of i) mitogen-activated protein kinases, ii) salicylic acid- and j asmonic acid-mediated defense signaling pathways, and iii) defense gene expression, and provide protection to a broad spectrum of pathogens (Manosalva, et al. (2015) Nature Comm., 6:7795; Klessig, et al. (2019) J. Phytopath., 167:265-272). Ascr #18 is the major ascaroside secreted by plant-parasitic nematodes. The results presented herein indicate that treatment of plants and/or seeds with ascr #18 enhances seed germination and/or promotes plant growth and/or yield. Since production of sufficient food and fiber from the earth's limited resources is dependent on the growth and ultimately on the yield from food and fiber, the present invention will also lead to enhanced food security.

In accordance with the instant invention, method for promoting, enhancing, and/or increasing plant growth are provided (e.g., promoting, enhancing, and/or increasing plant growth compared to untreated controls). In a particular embodiment, the method comprises contacting the target plant and/or its environment with an ascaroside (e.g., ascr #18) and/or a side-chain shortened analog or metabolite of the ascaroside (e.g., a side-chain shortened analog of ascr #18 such as ascr #9, ascr #10, and/or ascr #1). The compounds may be applied to any part of the plant (e.g., seed, root, and/or foliage) or plant cell and/or the nearby soil. The compounds may be applied to the plant at any stage of maturity (e.g., pre-germination, at seedling stage, at maturity, or any stage in between). The methods of the instant invention may be effective to result in one or more (or all) of the following: i) increased speed of germination, ii) decreased heterogeneity in the rate of of seed germination, iii) increased rate of growth of the roots, shoots, and/or plant (e.g., height), iv) reduced time to flowering and/or seed set and/or fruit or tuber set, and v) increased yield such as amount/weight of grain, weight of fruit, tubers, and/or total biomass. In a particular embodiment, the methods further comprise comparing the plant growth (e.g., one or more of the results set forth above) of the treated plant to untreated controls.

Any variety of plant (or plant part or cell) may be treated using the methods disclosed herein. Such plants include, without limitation, rice, tomato, Arabidopsis, tobacco, barley, potato, sweet potato, yam, soybean, strawberry, sugar beet, corn, wheat, rye, oats, sorghum, millet, canola, bean, pea, chickpea, lentil, apple, banana, pear, cherry, peach, plum, apricot, almond, grape, kiwi, mango, melon, papaya, walnut, hazelnut, pistachio, raspberry, blackberry, loganberry, blueberry, cranberry, orange, lemon, grapefruit, tangerine, lettuce, carrots, onions, broccoli, cabbage, avocado, cocoa, cassava, cotton, and flax. In certain embodiments, the plant is a crop plant. In certain embodiments, the plant is a grain. In certain embodiments, the plant is a vegetable crop. In certain embodiments, the plant is an ornamental. In certain embodiments, the plant is a fruit crop. In a particular embodiment, the plant is selected from the group consisting of Arabidopsis, rice, tomato, corn, wheat, corn, soybean, and potato.

With regard to the above methods, the compound produced by the pathogen may be a foreign (non-self) molecule or macromolecule to the plant being treated. Typically, the pathogen-produced compound is one that is secreted by the pathogen. In a particular embodiment, the pathogen-produced compound is a small molecule. The pathogen-produced compound applied according to the methods herein may be chemically synthesized, produced by fermentation, and/or isolated from the pathogen or a related organism (e.g., from a culture of the pathogen or a related organism).

In a particular embodiment, the pathogen-produced compound comprises the formula:

G-Lp-(C═O)—XR,

wherein G represents a moiety selected from a sugar, an amino acid, a nucleic acid, a combination of two or more of these, and a derivative of any of these; Lp represents an optionally unsaturated chain comprising n carbon atoms, wherein n is an integer from 4 to 40 inclusive (e.g., a fatty acid-like side chain); X represents O or NR^{y} (particularly O); R is selected from ═H, a metal ion, an optionally substituted moiety selected from C₁₋₁₂ aliphatic, C₁₋₁₂ heteroaliphatic, aromatic, heteroaromatic, and -G-Lp-(C═O)—XR (e.g., a dimer or oligomer); and R^{y} is ═H or an optionally substituted moiety selected from C₁₋₁₂ aliphatic, C₁₋₁₂ heteroaliphatic, aromatic, and heteroaromatic.

In a particular embodiment, G is an amino acid, a peptide, or a derivative thereof. In a particular embodiment, G is a sugar. In a particular embodiment, G is a sugar linked to Lp via a glycosidic bond. In a particular embodiment, G is a deoxy sugar, ascarylose, rhamnose, or a derivative thereof. In a particular embodiment, G has the formula

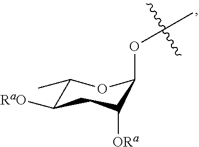

where each R^{a} is independently selected from H, alkyl, acyl, a glycoside, a peptide, or a nucleoside, particularly H. In a particular embodiment, G is ascarylose.

In a particular embodiment, R is a C₁₋₆ aliphatic. In a particular embodiment, R is H.

In a particular embodiment, Lp is an optionally substituted saturated or unsaturated carbon chain (e.g., aliphatic chain). In a particular embodiment, Lp is an optionally substituted saturated or unsaturated chain containing 4 to 40 carbon atoms in its main chain (e.g., excluding any carbon atoms present on methyl or other groups branching from the main linear chain). In a particular embodiment, Lp is an optionally substituted, saturated or unsaturated chain containing 4 to 6, 4 to 8, 6 to 10, 6 to 12, 8 to 16, 10 to 20, 12 to 24, 16 to 24, or 20 to 32 carbon atoms in its main chain. In a particular embodiment, Lp is an optionally substituted, saturated or unsaturated chain containing more than 8, more than 10, more than 12, more than 14, more than 18, or more than 24 carbon atoms in its main chain.

In a particular embodiment, Lp is an optionally substituted, saturated chain. In certain embodiments, Lp is an optionally substituted, saturated lipid containing 4 to 40 carbon atoms. In a particular embodiment, Lp is a saturated, optionally substituted chain containing 4 to 6, 4 to 8, 6 to 10, 6 to 12, 8 to 16, 10 to 20, 12 to 24, 16 to 24, or 20 to 32 carbon atoms in its main chain.

In a particular embodiment, Lp is a mono- or polyunsaturated, optionally substituted chain. In certain embodiments, Lp is a mono- or polyunsaturated, optionally substituted chain comprising 4 to 40 carbon atoms in its main chain. In certain embodiments, Lp is a mono-unsaturated, optionally substituted chain containing 4 to 6, 4 to 8, 6 to 10, 6 to 12, 8 to 16, 10 to 20, 12 to 24, 16 to 24, or 20 to 32 carbon atoms in its main chain. In certain embodiments, Lp is a polyunsaturated, optionally-substituted chain containing 4 to 6, 4 to 8, 6 to 10, 6 to 12, 8 to 16, 10 to 20, 12 to 24, 16 to 24, or 20 to 32 carbon atoms in its main chain. In certain embodiments, Lp is a mono-unsaturated, optionally-substituted chain containing 4 to 6, 4 to 8, 6 to 10, 6 to 12, 8 to 16, 10 to 20, 12 to 24, 16 to 24, or 20 to 32 carbon atoms in its main chain.

In certain embodiments, Lp is a chain substituted at the carbon attached to G. In certain embodiments, Lp is a chain bearing a C₁₋₁₂ optionally substituted aliphatic group on the carbon atom attached to G. In certain embodiments, Lp is a chain bearing a C₁₋₈, C₁₋₆, a C₁₋₄, or a C₁₋₃ optionally substituted aliphatic group on the carbon atom attached to G. In certain embodiments, Lp is a chain bearing an aliphatic moiety selected from the group of methyl, ethyl, n-propyl, i-propyl, n-butyl, sec-butyl, vinyl, allyl, ethynyl, or propargyl on the carbon atom attached to G. In a particular embodiment, Lp has the formula —CHCH₃(CH₂)_{y}—, wherein y is an integer from 3 to 39. In a particular embodiment, Lp has the formula —CHCH₃(CH₂)_{y}—, wherein y is an integer from 3 to 6, from 4 to 8, from 6 to 10, from 6 to 12, from 8 to 16, from 10 to 20, from 12 to 24, from 16 to 24, or from 20 to 32 carbon atoms in its main chain. In a particular embodiment, Lp has the formula —CHCH₃(CH₂)₈—. In a particular embodiment, Lp has the formula —CHCH₃(CH₂)₉—. In a particular embodiment, Lp has the formula —CHCH₃(CH₂)₁₀—. In a particular embodiment, Lp has the formula —CHCH₃(CH₂)₁₂—. In a particular embodiment, Lp has the formula —CHCH₃(CH₂)₇—. In a particular embodiment, Lp has the formula —CHCH₃(CH₂)₆— and X is O. In a particular embodiment, Lp has the formula —CHCH₃(CH₂)₈—; X is O; and R is H.

In certain embodiments, Lp is a chain having geminal disubstitution on the carbon atom attached to G. In certain embodiments, Lp has a formula —C(CH₃)₂(CH₂)_{y}—, where y is as defined in the embodiments and examples herein.

In a particular embodiment, Lp is an unsaturated chain. In certain embodiments, Lp is an unsaturated chain having 1 to 3 sites of unsaturation. In certain embodiments, Lp is a mono-unsaturated chain. In certain embodiments, Lp has the formula —CHCH₃(CH₂)ₐ—CH═CH—(CH₂)_{b}—, wherein a and b are independently integers from 0 to 20 and the sum of a and b is 2 to 30. In a particular embodiment, Lp has the formula —CHCH₃(CH₂)_{z}CH═CH—, where z is an integer from 1 to 18. In a particular embodiment, Lp has the formula —CHCH₃(CH₂)_{z}CH═CH—, where z is an integer from 1 to 4, from 4 to 6, from 6 to 8, from 4 to 12, from 6 to 12, from 10 to 20, from 12 to 24, or from 16 to 24. In a particular embodiment, Lp has the formula —CHCH₃(CH₂)₂CH═CH—. In a particular embodiment, Lp has the formula —CHCH₃(CH₂)₃CH═CH—. In a particular embodiment, Lp has the formula —CHCH₃(CH₂)₄CH═CH—. In a particular embodiment, Lp has the formula —CHCH₃(CH₂)₅CH═CH—. In a particular embodiment, Lp has the formula —CHCH₃(CH₂)₆CH═CH—. In a particular embodiment, Lp has the formula —CHCH₃(CH₂)₂CH═CH— and X is O. In a particular embodiment, Lp has the formula —CHCH₃(CH₂)₂CH═CH—; X is O; and R is H. In a particular embodiment, Lp has the formula —CHCH₃(CH₂)₄CH═CH— and X is O. In a particular embodiment, Lp has the formula —CHCH₃(CH₂)₄CH═CH—; X is O; and R is H.

In certain embodiments where Lp conforms to a formula with a substructure —CHCH₃CH₂ . . . , the chiral center (e.g. the underlined carbon atom in the substructure) is enantio-enriched. In certain embodiments, the chiral center is substantially enantiopure. In certain embodiments, the chiral center has the R configuration. In certain embodiments, the chiral center has the S configuration. In certain embodiments, the chiral center is present as a racemic (or diastereomeric) mixture.

In a particular embodiment, the pathogen-produced compound is an ascaroside. Examples of ascarosides suitable for the present invention include, but are not limited to:

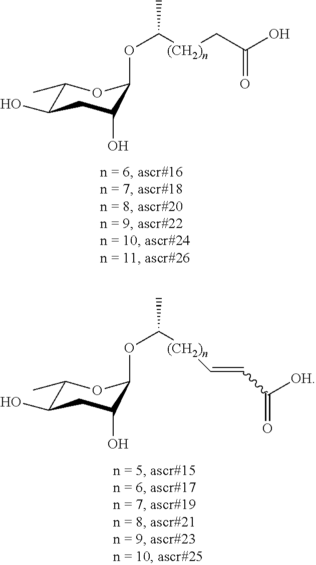

Further examples of ascarosides suitable for the present invention include, without limitation:

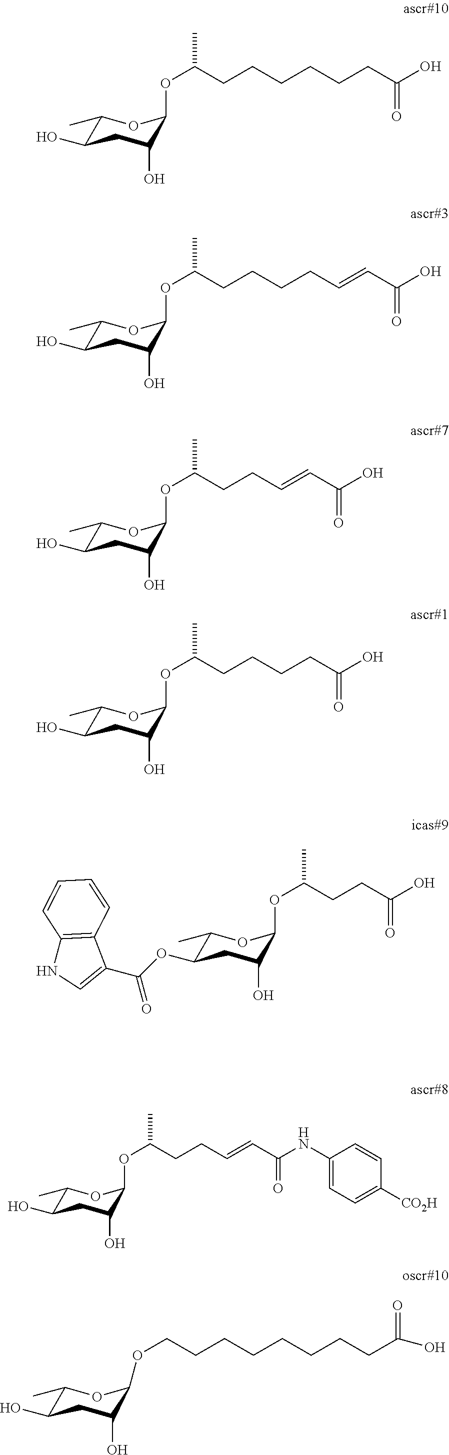

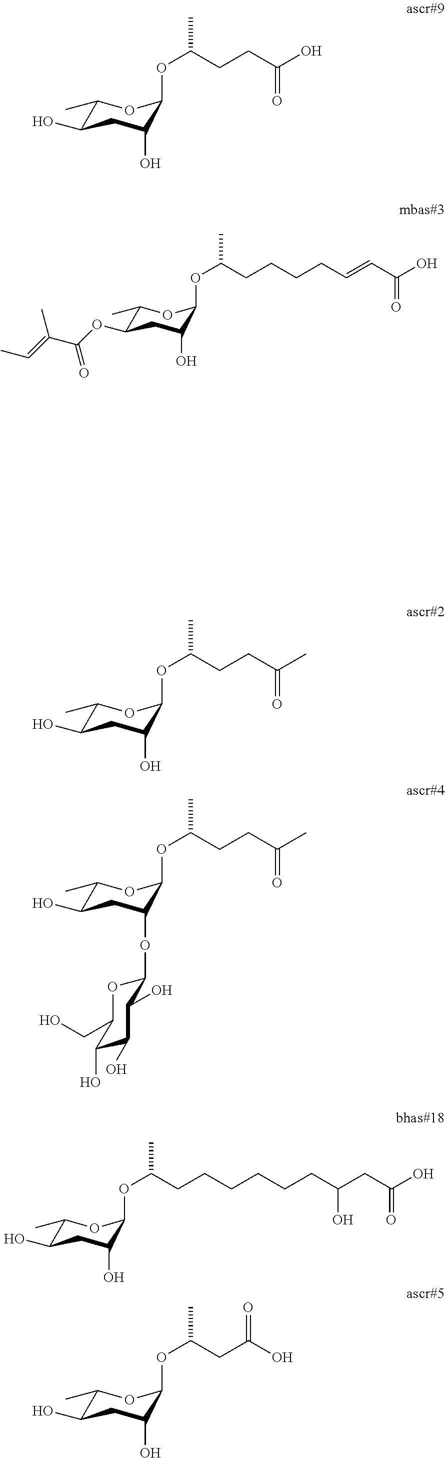

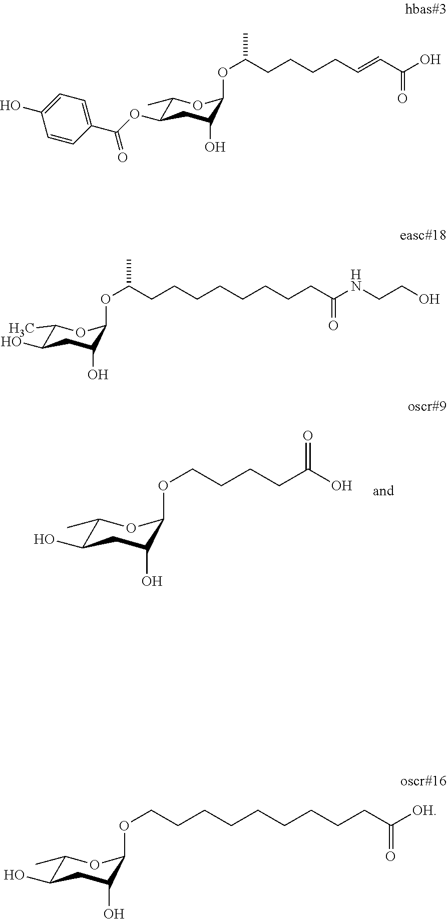

The instant invention also contemplates formulations and methods utilizing compounds that are structurally identical to the ascarosides depicted above except that the number of carbon atoms in the fatty acid-like side chain is changed (e.g., from between 3 and 32 carbons). Likewise, the instant invention encompasses compounds that are structurally identical to the ascarosides depicted above except that the identity of the substituents on the ascarylose oxygen atoms (e.g., on the hydroxyl groups at the 2- and 4- positions of the sugar) is changed. The instant invention also encompasses compounds that are structurally identical to the ascarosides depicted above except that the stereochemistry of one or more chiral centers is different (e.g., enantiomers, diastereomers or racemates of the depicted compounds). The instant invention also encompasses compounds that are structurally identical to the ascarosides depicted above except for the degree or pattern of deoxygenation of the sugar (e.g., compounds where one or both of the 3- and 6- positions of the sugar are not deoxygenated, and/or compounds where one or both of the 2- and 4- positions are deoxygenated). The instant invention also encompasses compounds that are structurally identical to the ascarosides depicted above except that the oxygen atom in the sugar ring is replaced by a carbon or nitrogen atom (e.g., replaced by —C(R^{y})₂— or by —NR^{y}—, where each Ry is independently as defined above and in the genera and subgenera herein).

In a particular embodiment, the pathogen-produced compound comprises ascr #18. In a particular embodiment, the pathogen-produced compound is a side-chain shortened metabolite or analog of ascr #18 (e.g., ascr #10, ascr #1, ascr #9). In a particular embodiment, the pathogen-produced compound is ascr #10, ascr #1, ascr #9, ascr #3, or ascr #18. In a particular embodiment, the pathogen-produced compound is a terminally substituted metabolite or analog of ascr #18. In a particular embodiment, the pathogen-produced compound is oscr #10 or oscr #16. In a particular embodiment, the pathogen-produced compound is a derivative of ascr #18. In a particular embodiment, the pathogen-produced compound is an unsaturated analog of an ascaroside (e.g., a sidechain unsaturated analog of ascr #10, ascr #1, ascr #9, ascr #3, or ascr #18). In certain embodiments, such derivatives comprise modification of the ascarylose. In certain embodiments, such derivatives comprise esters, thioesters, or amides of the fatty acid sidechain. In certain embodiments, such derivatives comprise dimers, trimers, oligomers or polymers of as ascaroside, (e.g. a dimer, trimer, oligomer, or polymer of ascr #10, ascr #1, ascr #9, ascr #3, ascr #18, or their derivatives).

As stated hereinabove, the methods of the instant invention comprise contacting the plant or its environment (e.g., its immediate environment (e.g., with regard to a plant, to the soil, particularly within the area of soil containing the root system of the plant or the plant seed)) with a pathogen-produced compound (e.g., ascaroside). The compounds of the instant invention may be administered to any part of the plant. For example, the compounds of the instant invention may be administered to a root, stem, leaf, seed and/or flower of the plant. In a particular embodiment, the compounds of the instant invention are administered to a root of the plant. In a particular embodiment, the compounds of the instant invention are administered to a seed of the plant. In a particular embodiment, the compounds of the instant invention are administered to seed grain of a plant intended to be planted for purposes of producing or propagating the plant. In a particular embodiment, the compounds of the instant invention are administered to a leaf of the plant.

The treatment of plants and/or soil with the compounds and formulations described herein may be carried out directly or by allowing the compounds to act on the surroundings, environment or storage space by the customary treatment methods, for example by immersion, spraying, evaporation, fogging, scattering, painting on and, in the case of propagation material, in particular in the case of seeds, also by applying one or more coats.

In a particular embodiment, the compounds are delivered to the plant in a sterile or nearly sterile environment (e.g., in the general absence of pathogens (e.g., nematode, virus, bacteria, fungus, insect, and/or oomycete, particularly in the absence of nematodes and bacteria). The effects of the instant invention are a direct increase of plant growth as opposed to better growth due to pathogen resistance. For example, the compounds may be delivered to the plant in a sterile or nearly sterile environment (e.g., in the general absence or exclusion of pathogens) to allow for rapid germination and/or plant growth (e.g., for at least one, two, three, or more weeks) prior to exposure to pathogens.

In a particular embodiment, the compounds of the instant invention are administered by foliar application. In a particular embodiment, the compounds of the instant invention are administered through the root system via the soil (systemic action) by drenching the locus of the plant with a liquid preparation or by incorporating the substances into the soil in solid form, e.g., in the form of granules comprising the substances compounded with carriers (soil application). In rice cultivations, these granules may be dispensed over the flooded paddy field. The compounds of the invention may also be applied to tubers or seed grain, for example, by soaking, spraying or drenching the seed grain or tubers in a liquid composition or by coating the tubers or seed grain with a solid composition. In a particular embodiment, the compounds are in a liquid composition which is sprayed onto the plant or plant part (e.g., tuber or seed).

The compounds of the instant invention may be used alone or contained in a composition with a carrier. For example, the compounds described herein may be formulated together with an agronomically acceptable carrier. The term “agronomically acceptable carrier” includes any carrier suitable for administration to a plant or soil. For example, customary excipients in formulation techniques, such as solutions (e.g., directly sprayable or dilutable solutions), aqueous solutions, emulsions, (e.g., emulsion concentrates and diluted emulsions), wettable powders, suspensions, soluble powders, powders, dusts, pastes, soluble powders, granules, suspension-emulsion concentrates, encapsulation into polymeric materials, coatable pastes, natural and synthetic materials impregnated with active compound and microencapsulations in polymeric substances. These formulations are produced in a known manner, for example by mixing the compounds with agronomically acceptable carrier, such as liquid solvents or solid carriers, optionally with the use of surfactants, including emulsifiers, dispersants, and/foam-formers. In a particularly embodiment, the agronomically acceptable carrier is synthetic or nan-natural.

If the agronomically acceptable carrier is water, the composition may also comprise auxiliary solvents such as organic solvents. Suitable liquid solvents include, for example, aromatics (e.g., xylene, toluene and alkylnaphthalenes); chlorinated aromatics or chlorinated aliphatic hydrocarbons (e.g., chlorobenzenes, chloroethylenes and methylene chloride); aliphatic hydrocarbons (e.g., cyclohexane); paraffins (e.g., petroleum fractions, mineral and vegetable oils); alcohols (e.g., butanol or glycol and also their ethers and esters); ketones (e.g., acetone, methyl ethyl ketone, methyl isobutyl ketone and cyclohexanone) and strongly polar solvents (e.g., dimethylformamide and dimethyl sulphoxide). It is preferred that nontoxic carriers be used in the methods of the present invention.

Suitable solid agronomically acceptable carriers include, for example, ammonium salts and ground natural minerals (e.g., kaolins, clays, talc, chalk, quartz, attapulgite, montmorillonite and diatomaceous earth); ground synthetic minerals (e.g., highly disperse silica, alumina and silicates); crushed and fractionated natural rocks (e.g., calcite, marble, pumice, sepiolite and dolomite); synthetic granules of inorganic and organic meals; and granules of organic material (e.g., sawdust, coconut shells, maize cobs, and tobacco stalks).

Suitable emulsifiers and foam-formers include, for example, nonionic and anionic emulsifiers (e.g., polyoxyethylene fatty acid esters, polyoxyethylene fatty alcohol ethers, for example, alkylaryl polyglycol ethers, alkylsulphonates, alkyl sulphates and arylsulphonates) and protein hydrolysates.

Suitable dispersants include, for example, lignin-sulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids, such as cephalins and lecithins, and synthetic phospholipids, can be also used in the formulations. Other additives may include, for example, mineral and vegetable oils.

Colorants such as inorganic pigments, for example, iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc may also be included in the agronomically acceptable carrier.

The compounds or compositions of the instant invention may be administered to the plant and/or soil by any techniques known in the art, including, for example, spraying, atomizing, dusting, scattering, coating or pouring. One of skill in the art would be able to determine the appropriate technique for administration without undue experimentation according to the specific chemical composition and formulation of the compound being employed, the method of applying the compound/formulation, and the locus of treatment.

The compositions disclosed herein generally comprise between 0.001 and 95% by weight of active compound(s), particularly between 0.001 and 1%. Favorable application rates are, in general, 0.001 g to 1,000 g of active substance(s) (AS) per hectare (ha), for example, 0.001 g to 0.01 g AS/ha, 0.01 g to 0.1 g AS/ha, 0.1 g to 0.5 g AS/ha, 0.5 g to 1 g AS/ha, 1 g to 5 g AS/ha, 5 g to 25 g AS/ha, 25 g to 100 g AS/ha, 100 to 500 g AS/ha, or 500 to 1,000 g AS/ha. For application of tubers or seed grain, dosages of 0.001 mg to 1,000 mg active substance per kg of seed grain or tubers may be used, for example or 0.001 to 0.01 mg/kg, 0.01 to 0.05 mg/kg, 0.05 to 0.1 mg/kg, 0.1 to 0.5 mg/kg, 0.5 to 1 mg/kg, 1 to 5 mg/kg, 5 to 10 mg/kg, 10 to 50 mg/kg, 50 to 500 mg/kg, or 500 to 1,000 mg/kg.

### Definitions

The following definitions are provided to facilitate an understanding of the present invention.

The singular forms “a,” “an,” and “the” include plural referents unless the context clearly dictates otherwise.

The term “ascaroside” refers to any of a group of glycolipids, containing the sugar ascarylose, found in nematodes.

The term “pathogen” refers to any bacterium, fungus, oomecyte, virus, nematode (e.g., cyst or root knot nematode), or insect, with pathogenic effects on the plant.

The term “substantially pure” refers to a preparation comprising at least 50-60% by weight of a given material (e.g., small molecule, nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC-MS analysis, and the like).

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl acetate, phosphate), antimicrobial, bulking substance (e.g., lactose, mannitol), excipient, auxiliary agent or vehicle with which an active agent of the present invention is administered. Carriers can be sterile liquids, such as water and oils, including those of petroleum, animal, vegetable or synthetic origin. Water or aqueous saline solutions and aqueous dextrose and glycerol solutions can be employed as carriers.

As used herein, the term “small molecule” refers to a substance or compound that has a relatively low molecular weight (e.g., less than 4,000, less than 2,000, particularly less than 1 kDa or 800 Da). Typically, small molecules are organic.

The term “aliphatic” refers to a non-aromatic hydrocarbon-based moiety. Aliphatic compounds can be acyclic (e.g., linear or branched) or cyclic moieties (e.g., cycloalkyl) and can be saturated or unsaturated (e.g., alkyl, alkenyl, and alkynyl). Aliphatic compounds may comprise a mostly carbon main chain (e.g., 1 to about 30 carbons) and comprise heteroatoms and/or substituents (see below). The term “alkyl,” as employed herein, includes saturated or unsaturated, straight or branched chain hydrocarbons containing 1 to about 30 carbons in the normal/main chain, particularly 24 or fewer carbon atoms (e.g., methyl, ethyl, n-propyl, ipropyl, n-butyl, s-butyl, t-butyl, n-pentyl, i-pentyl, n-hexyl, and the like). Branched alkyl means that one or more lower alkyl groups such as methyl, ethyl, or propyl are attached to a linear alkyl chain. The hydrocarbon chain of the alkyl groups may be interrupted with one or more heteroatom (e.g., oxygen, nitrogen, or sulfur). An alkyl (or aliphatic) may, optionally, be substituted (e.g. with fewer than about 8, fewer than about 6, or 1 to about 4 substituents). The term “lower alkyl” or “lower aliphatic” refers to an alkyl or aliphatic, respectively, which contains 1 to 3 carbons in the hydrocarbon chain. Alkyl or aliphatic substituents include, without limitation, alkyl (e.g., lower alkyl), alkenyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol.

“Alkenyl” means an alkyl, as defined above, containing at least one double bond between adjacent carbon atoms. Alkenyls include both cis and trans isomers. Branched alkenyl means that one or more lower alkyl groups such as methyl, ethyl, or propyl are attached to a linear alkenyl chain.

The term “halogen” refers to fluoro, chloro, bromo, and iodo. The term “halo alkyl” refers to a branched or straight-chain alkyl as described above, substituted with one or more halogens.

The term “acyl” refers to a group of general formula —C(O)R, wherein R is an aliphatic or alkyl. In a particular embodiment, the term “acyl” refers to groups of from 1 to 8 carbon atoms of a straight, branched, or cyclic configuration, saturated, unsaturated, or aromatic, and combinations thereof, attached to the parent structure through a carbonyl functionality. One or more carbons in the acyl residue may be replaced by nitrogen, oxygen, or sulfur as long as the point of attachment to the parent remains at the carbonyl. Examples include acetyl (Ac), benzoyl, propionyl, isobutyryl, t-butoxycarbonyl, benzyloxycarbonyl, and the like.

Amino acids can be in D- or L-configuration. Suitable amino acids include α-amino acids, (β-amino acids, γ-amino acids, δ-amino acids, and ε-amino acids, and include not only natural amino acids (i.e., those found in biological systems, including the twenty amino acids found in natural proteins), but also naturally-occurring variants of such amino acids, as well as synthetic amino acids and their analogues known to those skilled in the art. Exemplary amino acids include, without limitation: the twenty natural amino acids, 4-hydroxyproline, hydroxyysine, demosine, isodemosine, 3-methylhistidine, norvalin, beta-alanine, gamma-aminobutyric acid, citrulline, homocysteine, homoserine, ornithine, and methionine sulfone.

The term “aromatic” or “aryl” means an aromatic monocyclic or multi-cyclic (polycyclic) ring system of 6 to about 19 carbon atoms, for instance, about 6 to about 10 carbon atoms, and includes arylalkyl groups. Representative aryl groups include, but are not limited to, groups such as phenyl, naphthyl, azulenyl, phenanthrenyl, anthracenyl, fluorenyl, pyrenyl, triphenylenyl, chrysenyl, and naphthacenyl. The term “heteroaromatic” or “heteroaryl” means an aromatic monocyclic or multi-cyclic ring system of about 5 to about 19 ring atoms, for instance, about 5 to about 10 ring atoms, in which one or more of the atoms in the ring system is/are element(s) other than carbon, for example, nitrogen, oxygen, and/or sulfur. In the case of multi-cyclic ring systems, only one of the rings needs to be aromatic for the ring system to be defined as “heteroaromatic” or “heteroaryl”. Exemplary “heteroaromatic” or “heteroaryl” may contain about 5 or 6 ring atoms. Representative heteroaryls include, but are not limited to, purinyl, pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, quinolinyl, isoquinolinyl, quinazolinyl, cinnolinyl, pthalazinyl, quinoxalinyl, and the like.

The term “fatty acid” generally refers to a carboxylic acid with an aliphatic tail (chain). The aliphatic chain can be between about 2 and about 36 carbon atoms in length. Fatty acids can be saturated, unsaturated, or polyunsaturated. The aliphatic chain can be a linear or a branched chain. The term “fatty acid” may be used herein to refer to a “fatty acid derivative” which can include one or more different fatty acid derivatives, or mixtures of fatty acids derivatives. Exemplary fatty acids include, without limitation, unsaturated fatty acids, saturated fatty acids, and diacids; mono-, di-, and tri-glycerides of ascarosides that have a carboxylic acid functionality; hydroxy acids, co hydroxy acids, co-I hydroxy acids, di-hydroxy fatty acids (e.g., dihydroxy fatty acids that are omega- or omega-1 hydroxylated, as well as alpha- or beta-hydroxylated fatty acids).

The term “sugar” includes mono-, di-, tri-, and oligosaccharides. The sugar may be naturally occurring or synthetic. In a particular embodiment, the sugar is a monosaccharide. In a particular embodiment, the monosaccharide is cyclic. In a particular embodiment, the monosaccharide comprises 3-10 carbon atoms. The monosaccharide can be in D- or L- configuration. In a particular embodiment, the monosaccharide is a deoxy sugar. In certain embodiments, the term “sugar” also encompasses carbo-sugars and amino sugars where one or more oxygen atoms (particularly, the ring oxygen of a furanose or pyranose sugar) are replaced by a carbon or nitrogen atom respectively.

The following examples are provided to illustrate certain embodiments of the invention. They are not intended to limit the invention in any way.

### EXAMPLE 1

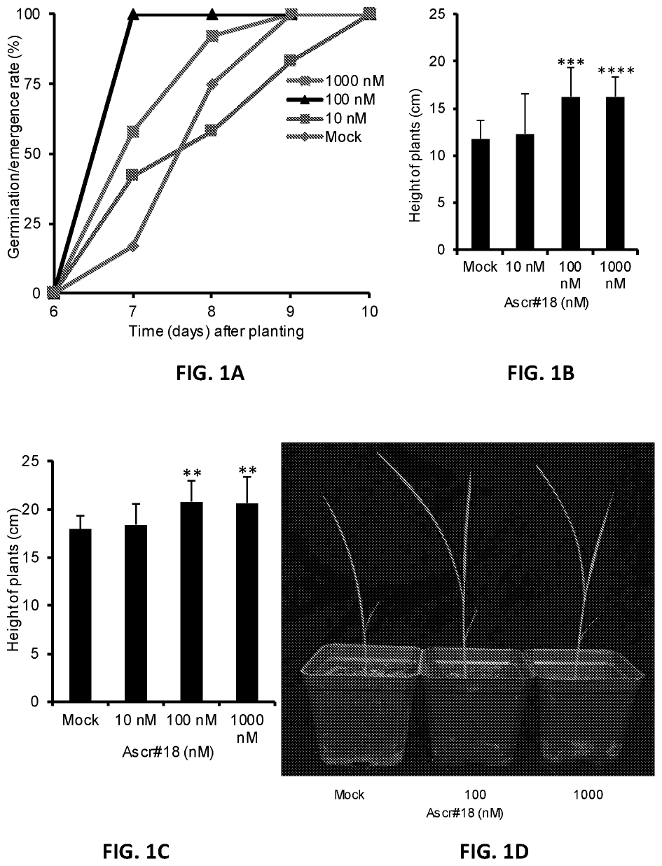

The effect of ascr #18 on rice seed germination/emergence and growth was tested. Briefly, rice seeds were pretreated 24 hours without (mock) or with different concentrations (10, 100 and 1000 nM) of ascr #18 before planting in soil. Seed germination/emergence from soil was measured after 7, 8, 9, 10 and 11 days after planting. As seen in FIG. 1A, increased amount of ascr #18 resulted in a faster germination/emergence rate for the rice seeds. The length of plants was also measured 15 days and 18 days after planting and pictures were taken at 15 days after planting. As seen in FIGS. 1B-1D, rice plants were taller with seed treatments with 100 nM or 1000 nM ascr #18 than untreated seeds or seeds treated with only 10 nM ascr #18.

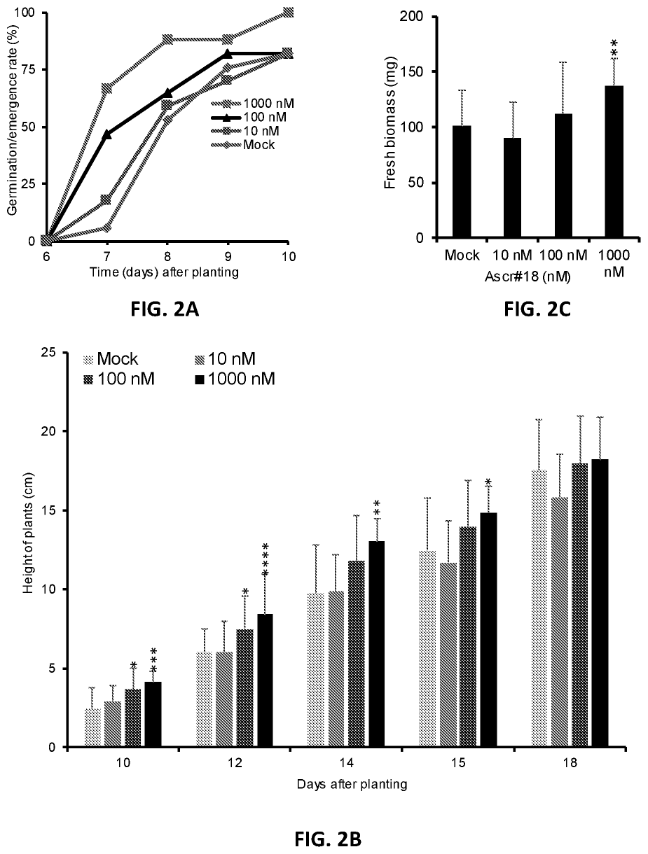

Further studies of the effect of ascr #18 on rice seed germination/emergence and growth were performed. Rice seeds were pretreated 24 hours without (mock) or with different concentrations (10, 100 and 1000 nM) of ascr #18 before planting in soil. Seed germination/emergence from soil was measured after 6, 7, 8, 9 and 10 days after planting. As seen in FIG. 2A, increased amount of ascr #18 resulted in a faster germination/emergence rate for the rice seeds. The length of plants was also measured at 10, 12, 14, 15 and 18 days after planting. As seen in FIG. 2B, increased amount of ascr #18 resulted in a faster growth for the rice plants. The fresh biomass of plants was also measured 18 days after planting. As seen in FIG. 2C, increased amount of ascr #18 resulted in a greater biomass for the rice plants.

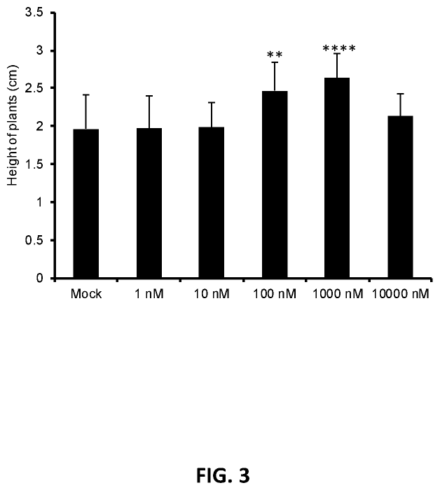

The effect of ascr #18 on was also determined on tomato plant growth. Tomato seeds were pretreated 24 hours without (mock) or with different concentrations (1, 10, 100, 1000 and 10000 nM) of ascr #18 before planting on soil. The length of the plants was measured 12 days after planting. As seen in FIG. 3, tomato plants were taller with seed treatments with 100 nM or 1000 nM ascr #18 than untreated seeds or seeds treated with only 10 nM ascr #18.

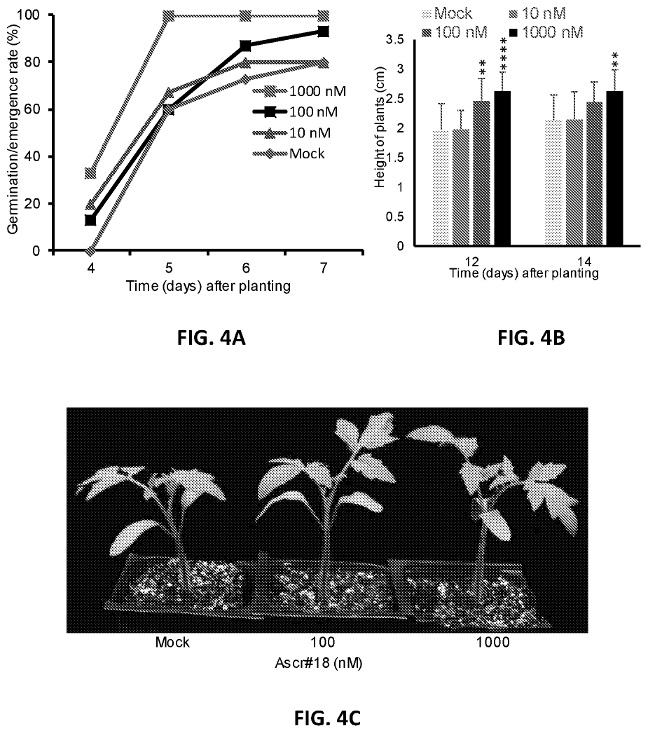

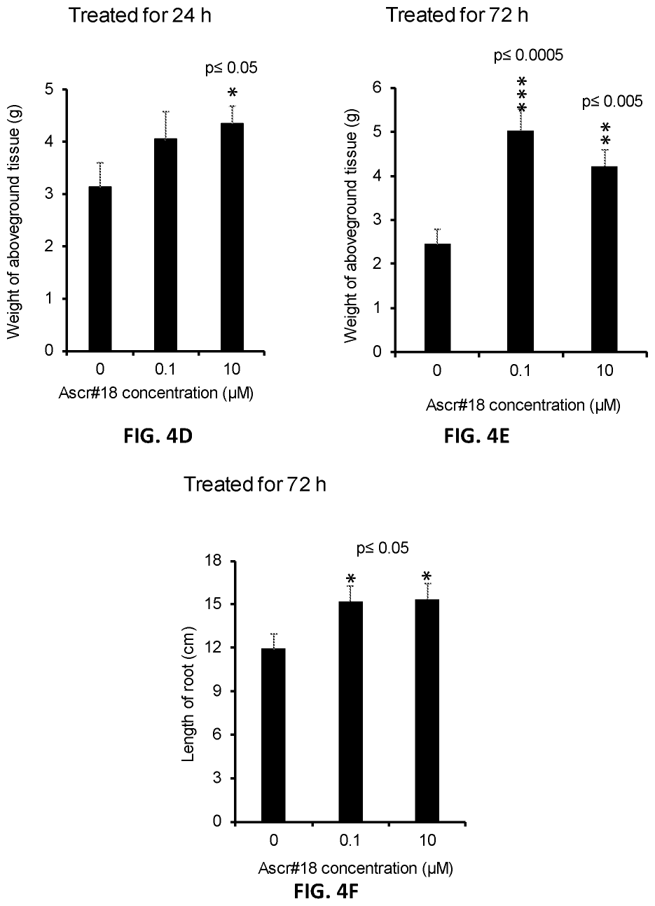

Further studies on the effect of ascr #18 on tomato seed germination/emergence and growth. Tomato seeds were pretreated 24 hours without (mock) or with different concentrations (10, 100, and 1000 nM) of ascr #18 before planting in soil. Seed germination/emergence from soil was measured after 4, 5, 6 and 7 days after planting. As seen in FIG. 4A, increased amount of ascr #18 resulted in a faster germination/emergence rate for the tomato seeds. The length of the plants was also measured 12 and 14 days after planting and pictures were taken 14 days after planting. As seen in FIGS. 4B-4C, tomato plants were taller with seed treatments of increasing amounts of ascr #18. In further experiments, tomato seeds were pretreated 24 or 72 hours without (mock) or with different concentrations (0.1 and 10 μM) of ascr #18 before planting in soil. More specifically, the tomato seeds were submerged in aqueous solutions of ascaroside for 24 hours or 72 hours directly before planting. Alternatively, seeds, (e.g., tomato seeds) can be coated with a solution of different concentrations of ascaroside containing polymeric binders commonly used for seed coating. Following treatment, the seeds can be dried and stored (e.g., for 2 weeks) before planting. The fresh weight of above-ground (excluding root) part of the plants was measured 21 days after planting. As seen in FIG. 4D-4E, ascr #18 treatment resulted in a greater weight for the tomato plants, particularly with longer pretreatment. The length of the roots of the plants was also measured 21 days after planting. As seen in FIG. 4F, tomato plants possessed longer roots with ascr #18 treatment compared to mock treated.

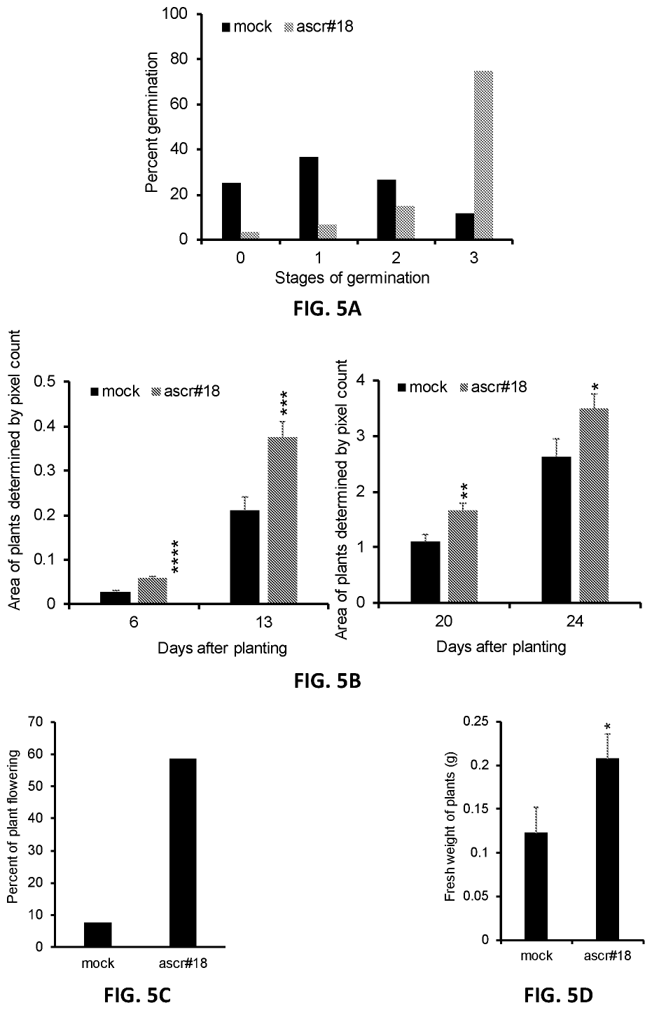

The effects of ascr #18 on Arabidopsis were also studied. Arabidopsis seeds were pretreated 24 hours without (mock) or with 1 μM ascr #18 for ascr #18 before planting in soil. The number of plants at different stages of germination were counted 3 days after planting. As seen in FIG. 5A, ascr #18 treatment resulted in a faster germination/emergence rate for the Arabidopsis seeds. Additionally, the surface area of germinated plants was measured by taking pictures and counting pixels using Fiji software. The data was then normalized by the total area for each plant. As seen in FIG. 5B, ascr #18 treatment resulted in greater plant surface area. The percentage of plants flowering at day 25 after planting in soil was also determined. As seen in FIG. 5C, ascr #18 treatment resulted in greater plant flowering. The fresh weight of above-ground (excluding root) part of the plants was also measured 25 days after planting. As seen in FIG. 5D, increased amount of ascr #18 resulted in a greater weight for the Arabidopsis plants.

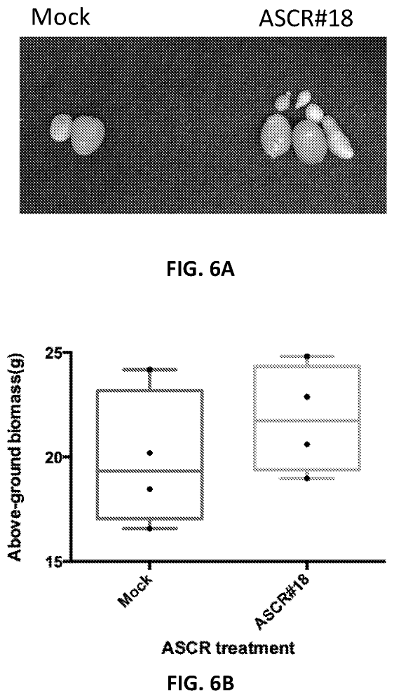

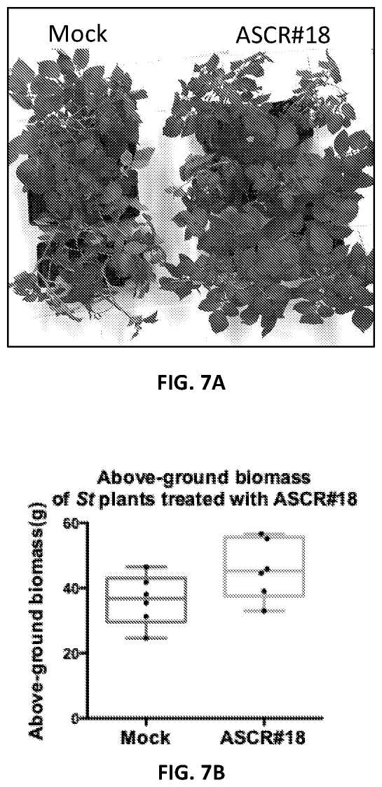

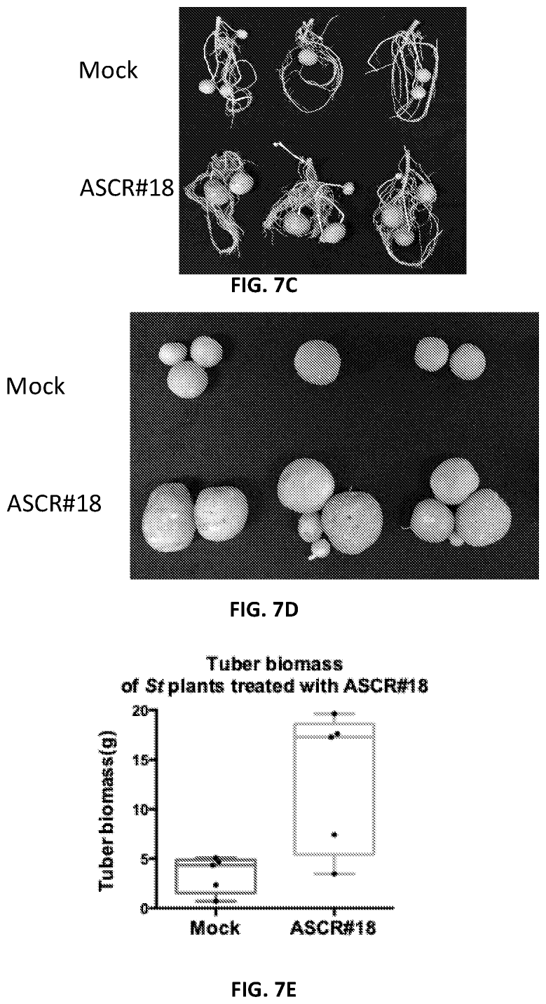

The effects of ascr #18 on potato were also studied. Four week old potato plants were pretreated (sprat treatment) without (mock) or with ascr #18 (10 nM). Potato tubers were harvested from mock and pre-treated plants. Each pile of tubers in FIG. 6A were from one plant. Ascr #18 treated potato plants yielded significantly more potato tubers. The above-ground (excluding root) part of the potato plants was also measured. As seen in FIG. 6B and FIGS. 7A through 7E, ascr #18 treated potato plants resulted in a greater biomass for the potato plants compared to mock treated.

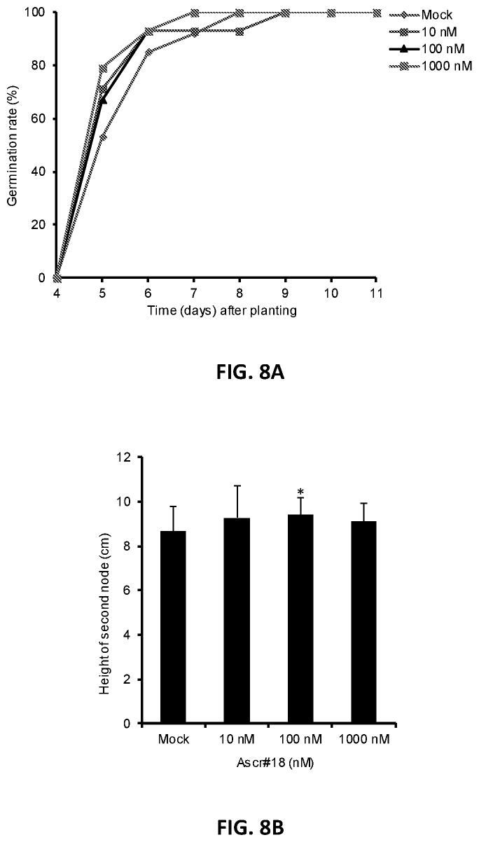

The effect of ascr #18 on corn seed germination/emergence and growth was also studied. Corn seeds were pretreated 24 hours without (mock) or with different concentrations (10, 100 and 1000 nM) of ascr #18 before planting in soil. Seed germination/emergence from soil was measured after 4, 5, 6, 7, 8, 9, 10 and 11 days after planting. As seen in FIG. 8A, ascr #18 treatment resulted in a faster germination/emergence rate for the corn seeds. The height of the second node of the plants was also measured 15 days after planting. As seen in FIG. 8B, corn plants were taller when seed were treated with ascr #18.

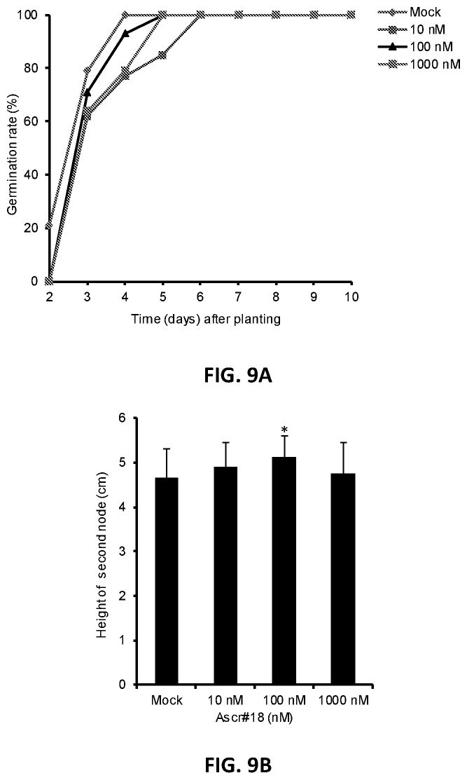

The effect of ascr #18 on wheat seed germination/emergence and growth was also studied. Wheat seeds were pretreated 24 hours without (mock) or with different concentrations (10, 100 and 1000 nM) of ascr #18 before planting in soil. Seed germination/emergence from soil was measured after 2, 3, 4, 5, 6, 7, 8, 9 and 10 days after planting (FIG. 9A). The height of the second node of the plants was also measured 10 days after planting. As seen in FIG. 9B, wheat plants were taller when seed were treated with ascr #18.

### EXAMPLE 2

Herein, an example of a synthesis protocol for ascr #18 is provided. The method may be modified to synthesize other ascarosides described herein. For example, the synthesis of ascr #18 metabolites such as ascr #1, ascr #10, and ascr #9 can be performed by replacing 7-bromoheptene in step 1 with a bromo containing compound having the correct number of carbons in the chain for the desired ascaroside.

### Synthesis of Ascr #18

Starting materials were synthesized as described in cited references or purchased from Sigma-Aldrich or Acros Organics and used without further purification. Anhydrous solvents were prepared with 4 Å molecular sieves. NMR spectra were recorded on a Varian INOVA-600 (600 MHz for ¹H, 151 MHz for ¹³C), INOVA-500 (500 MHz for ¹H and 125 MHz for ¹³C), and INOVA-400 (400 MHz for ¹H, 100 MHz for ¹³C) instruments. Flash chromatography was performed using a Teledyne ISCO CombiFlash system.

### Step 1. (9R)-hydroxydec-1-ene

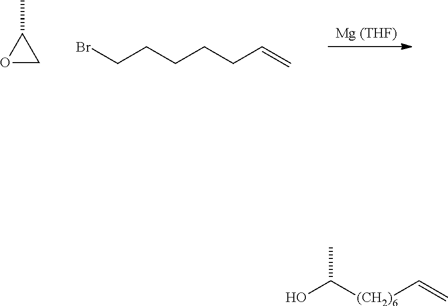

A solution of 7-bromoheptene (300 μg, 2 mmol) in dry THF (1 mL) was added drop wise to magnesium (240 mg, activated with iodine) in THF (500 μL). After stirring at RT for 1 hour the Grignard solution was transferred, cooled to −40° C. and treated with CuI (30 mg, 158 μmol). After stirring for 1 minute, (R)-propylene oxide (100 μL, 2 mmol) in THF (500 μL) was added and the solution stirred for 1.5 hours. The reaction was quenched with NH₄Cl (1 mL), extracted with diethyl ether, dried over Na₂SO4, and concentrated in vacuum. Flash column chromatography on silica gel using an ethyl acetate-hexane gradient (0 to 20%) afforded (8R)-hydroxydec-1-ene (56 mg, 359 μmol, 18% yield) as a colorless liquid. ¹H NMR (600 MHz, chloroform-d): δ1.18 (3H, d, J=6.2 Hz), 1.25-1.50 (10H, m), 2.01-2.07 (2H, m), 3.76-3.82 (1H, m), 4.91-4.95 (1H, m), 4.97-5.01 (1H, m), 5.81 (1H, ddt, J=17.1 Hz, 10.4 Hz, 6.7 Hz).

### Step 2. (9R)-(3′R,5′R-dibenzoyloxy-6′S-methyl-(2H)-tetrahydropyran-2-yloxy)-dec-1-ene

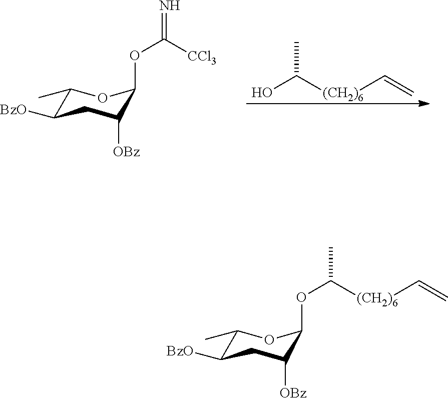

A solution of 2,4-di-O-benzoyl-ascarylose (Jeong et al. (2005) Nature 433:541-545) (139 mg, 390 μmol) in dry DCM (3 mL) was treated with trichloroacetonitrile (84 μL) and DBU (5 μL). After stirring at room temperature for 30 minutes, the solution was concentrated in vacuum. Flash column chromatography on silica gel using a mixture of ethyl acetate in hexane (20%) afforded (3′R,5′R-dibenzoyloxy-6′S-methyl-(2H)-tetrahydropyran-2-yloxy)-1-(2,2,2-trichloroacetimide) (152 mg, 302 μmol, 78%) as a colorless oil. A solution of 2,4-di-O-benzoyl-ascarylose-1-(2,2,2-trichloroacetimide) (152 mg, 302 μmol) in dry DCM (3 mL) at 0° C. was treated with (9R)-hydroxydec-1-ene (55 mg, 350 μmol) and trimethylsilyloxytriflate (5 μL). After 3 hours the solution was washed with saturated aqueous NaHCO₃ solution (0.5 mL), dried over Na₂SO₄ and concentrated in vacuum. Flash column chromatography on silica gel using a ethyl acetate-hexane gradient (5 to 20%) afforded (9R)-(3′R,5′R-dibenzoyloxy-6′S-methyl-(2H)-tetrahydropyran-2-yloxy)-dec-1-ene (91.1 mg, 184 μmol, 61%) as a colorless oil. ¹H NMR (400 MHz, chloroform-d): δ1.20 (3H, d, J=6.1 Hz), 1.30 (3H, d, J=6.1 Hz), 1.33-1.72 (10H, m), 2.09 (2H, m), 2.23 (1H, ddd, J=13.5 Hz, J=11.4 Hz, J=2.9 Hz), 2.44 (1H, m), 3.87 (1H, m), 4.15 (1H, dq, J=9.8 Hz, J=6.1 Hz), 4.95 (1H, ddt, J=10.2 Hz, J=2.2 Hz, J=1.3 Hz), 4.98 (1H, s.br), 5.02 (1H, ddt, J=17.1, Hz. J=2.2 Hz, J=1.6 Hz), 5.17 (1H, s.br), 5.21 (1H, ddd, J=10.3 Hz, J=4.6 Hz), 5.83 (1H, ddt, J=17.1 Hz, J=10.3 Hz, J=6.8 Hz), 7.45-7.51 (4H, m), 7.57-7.62 (2H, m), 8.06 (2H, m), 8.13 (2H, m); ¹³C NMR (100 MHz, chloroform-d): δ17.84, 19.14, 25.65, 28.84, 29.08, 29.38, 29.68, 33.76, 37.08, 66.89, 70.62, 71.21, 72.53, 93.72, 114.20, 128.38, 129.55, 129.80, 129.82, 129.96, 133.12, 133.17, 139.01, 165.59, 165.72.

### Step 3. Ethyl (10R)-(3′R,5′R-dibenzoyloxy-6′S-methyl-(2H)-tetrahydropyran-2-yloxy)-undec-2-enoate

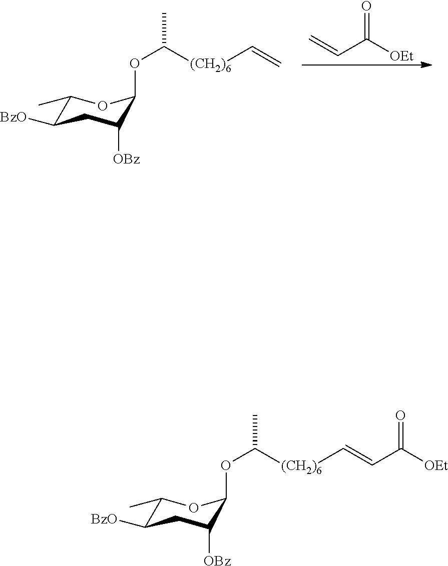

A solution of (9R)-(3′R,5′R-dibenzoyloxy-6′S-methyl-(2H)-tetrahydropyran-2-yloxy)-dec-1-ene (62 mg, 125 μmol) and ethyl propenoate (66 mg, 626 μmol) in DCM (5 mL) was treated with 1.4-benzoquinone (1.4 mg, 13 μmol) and Grubbs-II catalyst (5.3 mg, 6.3 μmol). After stirring at 40° C. for 15 hours, the reaction was filtered through a pad of silica using DCM:ethyl acetate (3:1). Flash column chromatography on silica gel using a ethyl acetate-hexanes gradient (10 to 50%) afforded ethyl (10R)-(3′R,5′R-dibenzoyloxy-6′S-methyl-(2H)-tetrahydropyran-2-yloxy)-undec-2-enoate (55 mg, 97 μmol, 78%) as a colorless oil. ¹H NMR (400 MHz, chloroform-d): δ1.19 (3H, d, J=6.1 Hz), 1.27 (3H, t, J=7.1 Hz), 1.28 (3H, d, J=6.3 Hz), 1.33-1.70 (10H, m), 2.16-2.26 (3H, m), 2.38-2.46, (1H, m), 3.84 (1H, m), 4.07-4.15 (1H, m), 4.17 (2H, q, J=7.1 Hz), 4.95 (1H, s.br), 5.12-5.23 (2H, m), 5.78-5.85 (1H. m), 6.97 (1H, dt, J=15.6 Hz, 7.0 Hz), 7.42-7.50 (4H, m), 7.55-7.62 (2H, m), 8.01-8.06 (2H, m), 8.09-8.14 (2H, m). ¹³C NMR (100 MHz, chloroform-d): δ14.42, 18.03, 19.30, 25.78, 28.16, 29.28, 29.53, 29.87, 32.32, 37.23, 60.29, 67.09, 70.80, 71.38, 72.78, 93.93, 117.65, 121.44, 128.58, 129.73, 129.98, 129.99, 130.13, 133.32, 133.38, 149.44, 165.80, 165.93, 166.89.

### Step 4. (10R)-(3′R,5′R-dihydroxy-6′S-methyl-(2H)-tetrahydropyran-2-yloxy)-undec-2-enoic Acid (ascr #17)

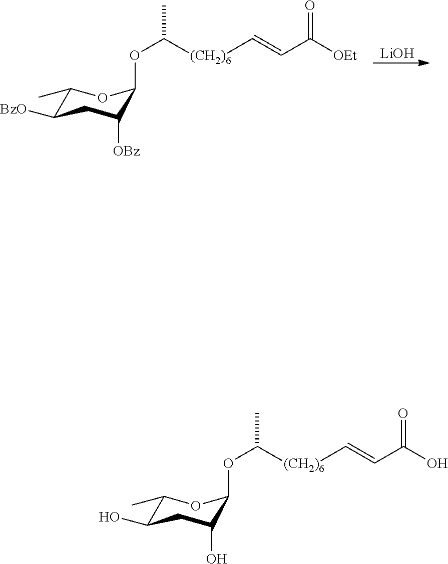

A solution of ethyl (10R)-(3′R,5′R-dibenzoyloxy-6′S-methyl-(2H)-tetrahydropyran-2-yloxy)-undec-2-enoate (55 mg, 97 μmol) in THF (1 mL) was added to a solution of lithium hydroxide (48 mg, 2 mmol) in water (380 μL) and 1,4-dioxane (2 mL). After stirring at 67° C. for 3 hours the mixture was neutralized with acetic acid and concentrated in vacuum. Flash column chromatography on silica gel using a methanol-dichloromethane gradient (0 to 30%) afforded (10R)-(3′R,5′R-dihydroxy-6′S-methyl-(2H)-tetrahydropyran-2-yloxy)-undec-2-enoic acid (ascr #17) (25.2 mg, 76.4 μmol, 79%) as a colorless oil. ¹H NMR (500 MHz, methanol-d₄): δ1.12 (3H, d, J=6.1 Hz), 1.21 (3H, d, J=6.3 Hz), 1.33 - 1.60 (10H, m), 1.76 (1H, ddd, J=13.3 Hz, J=11.4 Hz, J=3.1 Hz), 1.95 (1H, dt.br, J=13.1 Hz, J=4.1 Hz), 2.23 (2H, ddt, J=7.3 Hz, J=1.7 Hz, J=7.6 Hz), 3.52 (1H, ddd, J=11.3 Hz, J=9.5 Hz, J=4.6 Hz), 3.63 (1H, dq, J=9.3 Hz, J=6.4 Hz), 3.71 (1H, m), 3.78 (1H, m), 4.64 (1H, s.br), 5.80 (1H, dt, J=15.7 Hz, J=1.4 Hz), 6.95 (1H, dt, J=15.6 Hz, J=7.0 Hz); ¹³C NMR (100 MHz, methanol-d₄): δ18.27, 19.53, 26.95, 29.40, 30.40, 30.61, 33.29, 36.09, 38.51, 68.45, 70.10, 71.30, 72.62, 97.67, 122.75, 151.25, 170.37.

### Step 5. (10R)-(3′R,5′R-dihydroxy-6′S-methyl-(2H)-tetrahydropyran-2-yloxy)-undecanoic Acid (ascr #18)

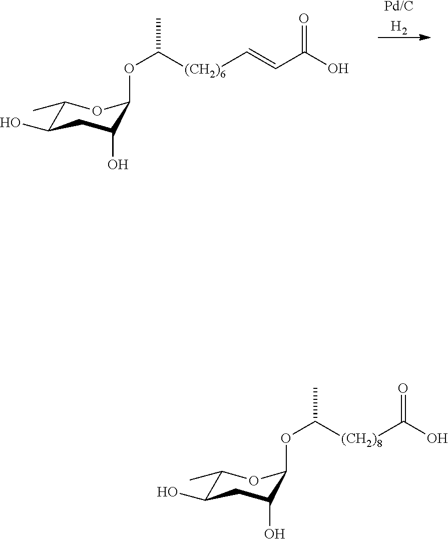

A solution of (10R)-(3′R,5′R-dihydroxy-6′S-methyl-(2H)-tetrahydropyran-2-yloxy)-undec-2-enoic acid (5 mg, 104 μmol) in methanol (1 mL) was treated with Pd/C (10% w/w) and hydrogenated for 14 hours. The solution was filtered and concentrated in vacuum to afford (10R)-(3′R,5′R-dihydroxy-6′S-methyl-(2H)-tetrahydropyran-2-yloxy)-undecanoic acid (4.4 mg, 76.4 μmol, 73%) as a colorless oil. ¹H NMR (500 MHz, methanol-d₄): δ1.12 (H, d, J=6.1 Hz), 1.21 (3H, d, J=6.3 Hz), 1.33 - 1.60 (14H, m), 1.76 (1H, ddd, J=13.3 Hz, J=11.4 Hz, J=3.1 Hz), 1.95 (1H, dt.br, J=13.1 Hz, J=4.1 Hz), 2.27 (2H, t, J=7.6 Hz), 3.52 (1H, ddd, J=11.3 Hz, J=9.5 Hz, J=4.6 Hz), 3.63 (1H, dq, J=9.3 Hz, J=6.4 Hz), 3.71 (1H, m), 3.78 (1H, m), 4.64 (1H, s.br); ¹³C NMR (100 MHz, methanol-d₄): δ18.11, 19.37, 26.40, 26.88, 30.37, 30.48, 30.61, 30.67, 35.97, 38.42, 68.34, 69.99, 71.17, 72.51, 97.56, 178.6.

Several publications and patent documents are cited throughout the specification in order to describe the state of the art to which this invention pertains. Each of these citations is incorporated herein by reference as though set forth in full.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of promoting, enhancing, and/or increasing plant growth, the method comprising the step of contacting the plant and/or its immediate environment with an effective amount of an ascaroside.

2. The method of claim 1, wherein the ascaroside is selected from the group consisting of ascr #9, ascr #10, ascr #1, ascr #3, and ascr #18.

3. The method of claim 1, wherein the ascaroside is ascr #18.

4. The method of claim 1, comprising treating a seed with the ascaroside.

5. The method of claim 1, comprising contacting the foliage or tubers of the plant with the ascaroside.

6. The method of claim 1, comprising contacting the soil near the plant or seed with the ascaroside.

7. The method of claim 1, wherein said plant is selected from the group consisting of Arabidopsis, rice, tomato, corn, wheat, corn, and potato.

8. The method of claim 1, comprising spraying a composition comprising the ascaroside onto a plant or plant part.

9. The method of claim 1, wherein the germination of the treated plant occurs faster than untreated plants.

10. The method of claim 1, wherein the rate of growth of the roots and/or shoots is faster than untreated plants.

11. The method of claim 1, wherein the time to flowering and/or seed set is reduced in treated plants compared untreated plants.

12. The method of claim 1, wherein the amount and/or weight of grain, tubers, and/or total biomass of the treated plant is increased compared to untreated plants.
